(19) United States
(12) Patent Application Publication
(10) Pub. No.: US 2026/0326104 A1
(43) Pub. Date: Oct. 22, 2026

(54) METHODS FOR PERFORMING BIOLOGICAL REACTIONS

(71) Applicant: Sphere Fluidics Limited, Cambridge (GB)

(72) Inventors: Frank F. Craig, Bury St Edmunds (GB); Serena A. Davoli, Cambridge (GB); Frank Gesellchen, Haverhill (GB); Xin Liu, Saffron Walden (GB); Marian Rehak, Cambridge (GB); Elena Shvets, Sawston (GB)

(73) Assignee: Sphere Fluidics Limited, Cambridge (GB)

(21) Appl. No.: 19/271,325

(22) Filed: Jul. 16, 2025

Related U.S. Application Data
(63) Continuation of application No. 16/954,553, filed on Dec. 19, 2018, now abandoned.

(30) Foreign Application Priority Data
Dec. 19, 2017 (GB) 1721344.8

(51) Int. Cl. B01L 3/00; B01L 3/02; C12M 1/12; C12M 1/42; C12M 3/06; C12N 13/00; C12N 15/87; C12N 15/88; C12Q 1/02

(52) U.S. Cl. CPC B01L 3/502784; B01L 3/0241; B01L 3/502761; C12M 23/16; C12M 25/01; C12M 35/02; C12M 35/04; C12N 13/00; C12N 15/87; C12N 15/88; C12Q 1/02; B01L 2200/0647; B01L 2200/16; B01L 2300/047; B01L 2300/0627

(57) ABSTRACT

A method of performing a biological reaction in a microfluidic droplet, and in particular to a method of performing genome editing in a microfluidic droplet. The invention also relates to microfluidic systems and instrumentations or products for performing these reactions.

### CROSS-REFERENCE TO RELATED APPLICATIONS

This application is a continuation application of U.S. patent application Ser. No. 16/954,553, filed on Jun. 17, 2020, which is a U.S. national stage filing, under 35 U.S.C. § 371(c), of International Application No. PCT/GB2018/053695, filed on Dec. 19, 2018, which claims priority to United Kingdom Patent Application No. 1721344.8, filed on Dec. 19, 2017. The entire contents of each of the aforementioned applications are incorporated herein by reference.

### FIELD OF THE INVENTION

The invention relates to a method of performing a biological reaction in a microfluidic droplet, and in particular to a method of performing genome editing in a microfluidic droplet. The invention also relates to microfluidic systems and instrumentations or products for performing these reactions.

### BACKGROUND OF THE INVENTION

Genome or gene editing uses targeted nucleases to specifically insert, delete or substitute DNA in an organism's genome. As such, genome editing has a substantial number of applications from gene therapy, drug discovery, neuroscience and disease modelling to agricultural and environmental applications. While there are a number of techniques for performing genome editing, it is the CRISPR-Cas9 model that has generated most excitement.

However, current techniques for performing gene editing, and in particular CRISPR-Cas require labour intensive and time-consuming operational steps, with significant reagent and material costs. There is therefore a need to be able to perform gene editing and other biological reactions faster, and with fewer reagents and materials. The present invention addresses this need.

### SUMMARY OF THE INVENTION

In one aspect of the invention, there is provided a method of performing genome editing in a microfluidic droplet, the method comprising: providing at least one microfluidic droplet, wherein said droplet comprises a cell or cell fragment, or nucleic acid derived therefrom, and genome editing reagents; and culturing the at least one droplet for sufficient time to perform genome editing in the cell or cell fragment.

Although some preferred implementations of the method employ cells or cell fragments, the method may also be used with nucleic acid derived from cells or cell fragments. The method may further comprise providing at least a first and a second droplet, wherein said first droplet comprises a cell or cell fragment, or nucleic acid derived therefrom, and the second droplet comprises genome editing reagents. The method may then further comprise fusing the first and second droplets, and culturing the fused droplet.

Thus in some implementations the method comprises providing at least two droplets, a first and a second droplet. The genome editing reagents may comprise at least one target DNA-binding reagent, at least one nuclease, and preferably a transfection or transduction or transformation reagent (such terms can be used interchangeably herein). The cell or cell fragment (or nucleic acid derived therefrom) and genome editing reagents may be distributed between the at least two droplets such that the cell or cell fragment (or nucleic acid derived therefrom) and genome editing reagents are not all present simultaneously in a single droplet. The method may then further comprise fusing the first and second droplets such that the cell or cell fragment (or nucleic acid derived therefrom) and genome editing reagents (target DNA-binding reagent, nuclease, and transfection or transduction reagent) are present simultaneously in a fused droplet. The fused droplet may then be cultured to perform genome editing in the cell or cell fragment.

Use of a transfection or transduction agent in this system is preferable but not essential; for example because a physical mechanism for transfection or transduction such as electroporation or squeezing could be employed.

In some implementations the method comprises providing at least a first, second and third droplet, wherein the first droplet comprises a cell or cell fragment (or nucleic acid derived therefrom), the second droplet comprises a target DNA-binding reagent and the third droplet comprises a nuclease, wherein the method comprises fusing the first, second and third droplets and culturing the fused droplet.

Thus in some implementations the method comprises providing at least a first, second, third and fourth droplet, wherein the first droplet comprises at least one cell or cell fragment (or nucleic acid derived therefrom), the second droplet comprises at least one target DNA-binding reagent, the third droplet comprises at least one nuclease and the fourth droplet comprises a transfection or transduction reagent, wherein the method comprises fusing the first, second, third and fourth droplets and culturing the fused droplet.

In another aspect of the invention, there is provided a method of performing genome editing in a microfluidic droplet, the method comprising: providing at least a first microfluidic droplet, wherein said droplet comprises a cell or cell fragment; injecting genome editing reagents into said droplet; and culturing the at least one droplet for sufficient time and under suitable conditions to perform genome editing in the cell or cell fragment.

In one embodiment, the droplet or first droplet comprises a single cell or cell fragment. In a further embodiment, the droplet or first droplet further comprises cell culture medium.

In another embodiment, the droplet is further cultured for sufficient time to allow cell division. Preferably, the droplet is cultured for at least 24 hours. More preferably, the droplet is cultured for between 48 and 72 hours.

In one embodiment, the droplet or the first, second or third droplet comprises a transfection or transduction reagent. In one example, the transfection or transduction reagent is Lipofectamine. Alternatively, the method further comprises transfecting the genome editing reagent into said cell, wherein preferably transfection or transduction is by membrane-disruption, selected from physical, mechanical, electrical, thermal and optical techniques.

In a further embodiment, the method further comprises preparing the at least one microfluidic droplet from at least one reservoir, as defined herein.

In another aspect of the invention, there is provided a method of reacting a biomolecule with a single biological entity, the method comprising providing a plurality of biological entities in a first fluid, providing a plurality of biomolecules in a second fluid, preparing at least one microfluidic droplet from the first and second fluid, wherein the droplet comprises a single biological entity and at least one biomolecule and culturing the at least one droplet for sufficient time to perform a reaction.

In one embodiment, the method comprises providing a plurality of biomolecules in a plurality of fluids, selecting at least one of the plurality of fluids and preparing at least one microfluidic droplet from the first fluid and the selected fluid(s), wherein the droplet comprises a single biological entity and at least one biomolecule.

In a further embodiment, the method comprises preparing a first and at least a second droplet, wherein said first droplet comprises at least one biological entity and said second droplet comprises at least one biomolecule, wherein the method further comprises fusing said first and at least said second droplets and culturing the fused droplet.

Preferably, the biomolecule is selected from the group comprising nucleic acids, polypeptides and peptides, ribonucleoproteins, a protein-nucleic acid complex, beads, lipids, nanoparticles, liposomes, micelles, sugars, carbohydrates, glycoproteins, microbes, viruses or viral-like particles, cell secreting modification and/or an engineering reagents, polymers, polymersomes, molecular imprinted polymers, polymer complexes, dendrimer, scaffolds, chromosomes, chromosome fragments, enzymes, chromosome/protein/chromatin complexes, aptamers, affimers and other non-antibody binding proteins/molecules, small molecules, therapeutics, organisms and transfection or transduction reagents.

Preferably, the biological entity is selected from the group comprising molecules, macromolecules, catalysts, viruses, prions, microbes, cells or cell fragments and organisms.

In one embodiment, the method further comprises determining whether the droplet or first droplet comprises no cells, one cell or a plurality of cells and sorting the droplet on the basis of the determination, wherein droplets with no or a plurality of cells are preferably passed to a waste outlet. Preferably the method comprises determining whether the droplet comprises no cells, one cell or a plurality of cells and said sorting the droplet is performed prior to culturing the droplet.

In another embodiment, the method further comprises analysing the droplet or fused droplet for a predetermined property following culturing of the droplet. Preferably the method further comprises sorting the droplet or fused droplet dependent on the analysis.

In a further embodiment, the method further comprises splitting said droplet or fused droplet into at least a first and second daughter droplet. Preferably, the first and second daughter droplets comprise at least one cell. More preferably, the method further comprises dispensing the droplet or daughter droplets. Even more preferably, the method further comprises analysing the dispensed droplet.

In one embodiment, the fusion is passive or active.

Preferably, passive fusion is performed by altering surfactant concentration, altering droplet surface tension, reducing the volume of oil between droplets, electrocoalescence, by electrically charging at least one droplet for fusing by electrostatic attraction or by physical constriction or physical collision.

Preferably, active fusion is performed using electric fields, lasers, acoustics, thermal energy or physical forces.

In another aspect of the invention there is provided a microfluidic system for reacting a biomolecule with a single biological entity, the system comprising at least one reservoir or channel, wherein the at least one reservoir (or channel) comprises a plurality of biological entities and biomolecules and an oil reservoir, a droplet formation device for preparing at least one droplet from the at least one reservoir (or channel) and oil reservoir and an incubator for culturing the droplet for sufficient time to perform a reaction between the biological entity and the biomolecule.

In a further aspect of the invention there is provided a microfluidic system for performing genome editing in a microfluidic droplet, the system comprising at least one reservoir, wherein the at least one reservoir comprises a plurality of cells and genome editing reagents and an oil reservoir, at least one droplet formation device for preparing at least one droplet from the at least one reservoir, and the oil reservoir and an incubator for culturing the droplet for sufficient time to perform genome editing.

In one embodiment, the system comprises a plurality of reservoirs, wherein each reservoir comprises a plurality of cells or cell fragments and genome editing reagents, wherein the cells of one reservoir are a different cell type or from a different sample source to the cells of at least one other reservoir.

In an alternative embodiment, the system comprises at least two reservoirs, wherein the first reservoir comprises a plurality of cells or cell fragments and the second reservoir comprises genome editing reagents. Preferably, one or more droplet formation devices prepare at least one droplet from the first and second reservoir, and the oil reservoir.

In a further embodiment, the system comprises at least three reservoirs, wherein the first reservoir comprises a plurality of cells or cell fragments, the second reservoir comprises genome editing reagents and the third reservoir comprises transfection or transduction reagents. Preferably, one or more droplet formation devices prepare at least one droplet from the first, second and third reservoir, and the oil reservoir.

In another aspect of the invention there is provided a microfluidic system for performing genome editing in a microfluidic droplet, the system comprising a first reservoir comprising a plurality of cells or cell fragments, a second reservoir comprising genome editing reagents, at least one oil reservoir, a first droplet formation device for preparing at least one droplet from the first reservoir and the oil reservoir, a second droplet formation device for preparing at least one droplet from the second reservoir and the oil reservoir, a droplet fusion region for fusing the at least one droplet prepared from the first and second droplet generation device and an incubator for culturing the droplet for sufficient time to perform genome editing. Preferably, in a further aspect of the invention there is provided a system to implement the method for performing genome editing described herein.

In one embodiment, the system further comprises at least one droplet sorting region for sorting a droplet based on one or more predetermined properties of the droplet. In a further embodiment, the system comprises two droplet sorting regions, a first droplet sorting region for sorting droplets that contain no or a plurality of cells and a second droplet sorting region for sorting droplets based on a predetermined property of the cell or cell fragment. Preferably, the first droplet sorting region is downstream of the droplet formation device. More preferably, the second droplet sorting region is downstream of the incubator.

In one embodiment, the system further comprises a droplet splitting region for splitting a droplet into at least two daughter droplets. Preferably, the system further comprises a droplet dispensing region for dispensing said sorted and/or split droplets.

In one embodiment, the system further comprises a droplet analyser, for analysing at least one predetermined property of at least one daughter droplet. Preferably, the droplet analyser comprises one or more of a fluorescence detector, a scattered light detector, an imaging detector, an acoustic wave generating and detecting unit and a magnetic activated cell sorting device.

In one embodiment, the genome editing reagents comprise at least one target DNA-binding reagent and at least one nuclease.

In one embodiment, the target DNA-binding reagent comprises a sgRNA nucleic acid or a sgRNA molecule. Preferably, the target DNA-binding reagent comprises a nucleic acid construct comprising a sgRNA nucleic acid operably linked to a regulatory sequence. More preferably, the nuclease is a Cas enzyme.

In an alternative embodiment, the target DNA-binding reagent comprises a TAL-effector DNA binding domain, and the nuclease comprises a DNA cleavage nuclease.

In a further alternative embodiment, the target DNA-binding reagent comprises a zinc finger DNA-binding domain and the nuclease is a DNA cleavage nuclease.

In one example, the transfection reagent is Lipofectamine. In an alternative embodiment, the system further comprises a transfecting region for transfecting the genome editing reagent into a cell, wherein preferably transfection or transduction is by membrane-disruption, selected from physical, mechanical, electrical, thermal and optical techniques.

In another aspect of the invention there is provided a microfluidic product, which comprises a substrate comprising at least one sample input channel for receiving a fluid comprising a plurality of biological entities and biomolecules, an oil input channel for receiving an oil, wherein the at least one sample and oil channels are fluidly connected to a droplet generating region for generating microfluidic droplets comprising at least one biomolecule and at least one biological entity, an incubator for culturing the droplet and at least one output channel.

In a further aspect of the invention, there is provided a microfluidic product comprising a substrate comprising a first input channel for receiving a fluid comprising a plurality of cell or cell fragments and optionally genome editing reagents, a second input channel for receiving an oil, wherein the first and second input channels are fluidly connected to a first droplet generating region for generating microfluidic droplets comprising at least one cell or cell fragment and optionally genome editing reagents, an incubator for culturing the droplet and at least one output channel.

In one embodiment, the product comprises a first inlet channel for receiving a fluid comprising a plurality of cell or cell fragments, a second input channel for receiving an oil, a third inlet channel for receiving genome editing reagents, and optionally a fourth input channel for receiving an oil. Preferably, the third input channel and second or optionally fourth channel are fluidly connected to a second droplet generating region, and wherein the microfluidic product further comprises a droplet fusion region for fusing at least one droplet prepared from the first droplet generation region with at least one droplet prepared from the second droplet generation region. In this embodiment, the first droplet generating region generates at least one microfluidic droplet from the first and second input channel, and the second droplet generating region generates at least one microfluidic droplet from the third and second or optionally the third and fourth input channels.

In one embodiment, the input channels, droplet generating regions, incubator, droplet fusion region and at least one outlet channel are incorporated on a single substrate.

In a further embodiment, the product further comprises a single cell sorting region, wherein preferably said single cell sorting region is downstream of the droplet generating region(s).

In a further embodiment, the product further comprises a sorting region where the droplet is analysed for a predetermined property or a phenotype and wherein the droplet is sorted dependent on the analysis. Preferably, the sorting region is downstream of the incubator.

In one embodiment, the product further comprises a droplet splitting region connected to the at least one output channel.

In another embodiment, the product is made from fluorinated or non-fluorinated plastics, glass, silicon or synthetic polymers. Preferably, the polymer is selected from the group comprising polydimethylsiloxane, polyurethane and styrene-ethylene-butadiene-styrene.

In a further aspect of the invention, there is provided a microfluidic droplet for performing genome editing, the droplet comprising at least one cell or cell fragment, cell culture medium and genome editing reagents, wherein the size of the droplet is between 100 and 10,000 pL and, wherein the droplet can be cultured for sufficient time for genome editing to occur in the encapsulated cell or cell fragment.

### DESCRIPTION OF THE FIGURES

These and other aspects of the invention will now be described, by way of example only, with reference to the accompanying figures in which:

FIG. 1 shows a schematic illustration of a microfluidic workflow for performing a biological reaction according to embodiments of the present invention.

FIG. 2 shows encapsulation of different types of adherent cells in picodroplets and quantification of their subsequent survival in picodroplets for prolonged period of time (5 days).

FIG. 3 shows that droplet generation and stability (24 h, 37 °C) is not affected by presence of different transfection reagents.

FIG. 4 shows that cells can be transfected in picodroplets using different transfection reagents.

FIG. 5 shows quantification of transfection efficiency of GFP expressing cells.

FIG. 6 shows representative example of experiment testing transfection efficiency of cells using classical transfection method or our in-house developed method of transfection in picodroplets.

FIG. 7 shows summary of experiments testing transfection efficiency using classical transfection method or our in-house developed method of transfection in microfluidic system (picodroplets or continuous microfluidics).

FIG. 8 shows a number of examples of different microfluidic workflows.

FIG. 9 shows images of droplets pre-sorting in bright field (a) and green fluorescence (b) of non-sorted droplets containing a positively edited HCT116 Fire line cell and gene editing reagents after 48 h at 37 °C. Arrow indicates the cell inside a droplet. (c) shows a comparison of GFP expression at 48 hours post-transfection using standard genome editing (bulk) versus genome editing in picodroplets.

FIG. 10 shows a scatter plot of the fluorescent signal from droplets from FIG. 9. Droplets have been re-injected into the system described herein and detected after 48 h at 37 °C. Polygon shows positive droplets (containing green-fluorescent cells) (FIG. 10a). Calculation of percentage of positive cells is done taking into consideration the input cell concentration and the predicted droplets occupancy, which follows Poisson distribution (see FIG. 10b).

FIG. 11 shows a plate scan of green fluorescent colonies at week 3. Arrows show some colonies. Imaging via plate imager (FIG. 11a). Higher resolution imaging of two colonies from the same plate (FIG. 11b), taken via fluorescent microscope. Images show the same two colonies and in bright field (right) and green fluorescence (left). FIG. 11c shows a table of colony outgrowth after dispensing of the edited cells (expressing GFP). The table shown in FIG. 11c shows the analysis of 96-well plates (n=6) collected in three separate experiments.

FIG. 12 shows genome editing of HCT 116 cells in picodroplets. 400 pL picodroplets containing HCT 116 cells and viral particles for genome editing after 18 h incubation (a and b). (c) shows a single picodroplet with multiple cells dispensed into a 96-well plate. (d to g) show micrographs of Cas9-GFP and mCherry tagged Action after successful genome editing. FIGS. 12 (h) and (i) show a y-junction chip.

### DETAILED DESCRIPTION OF PREFERRED EMBODIMENTS

Some example implementations of the present invention will now be further described. In the following passages, different aspects and embodiments of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The techniques described herein may be implemented using, unless otherwise indicated, conventional techniques of molecular biology, chemistry, biochemistry and recombinant DNA technology, bioinformatics which are within the skill of the art. Such techniques are explained fully in the literature.

As outlined in the summary part of the description, in some embodiments the method and system allow for the formation of a single droplet from at least one inlet that comprises a plurality of biological entities, such as cells or cell fragments and a plurality of biomolecules such as genome editing reagents. Advantageously, this method and system allows for faster droplet production rates and consequently a higher through-put rate. This method is also simpler than preparing multiple droplets containing the biological entities and biomolecules separately and fusing the droplets.

In other embodiments, as also described above, the system and method allow for the formation of at least two droplets that are subsequently fused. In this embodiment, one droplet comprises a biological entity, such as cells or cell fragments and another droplet comprises a plurality of biomolecules such as genome editing reagents. Advantageously, this method and system allows a fused droplet to be generated with a specific combination of biological entities and biomolecules. In other words, this method allows greater control over the final content of the droplet that is subsequently cultured and analysed. This embodiment also allows for a screening step to be performed before fusion to ensure each droplet contains only a single biological entity, such as a cell, before the droplet is fused with a second droplet containing a biomolecule. This embodiment may also be useful where the purpose is to screen a genome editing library, since each second droplet may contain a different member of the library.

Accordingly, in one embodiment of the invention, there is provided a method of reacting a biomolecule with a single biological entity, the method comprising providing a plurality of biological entities in a first fluid, providing a plurality of biomolecules in a second fluid, preparing at least one microfluidic droplet from the first and second fluid, wherein the droplet comprises a single biological entity and at least one biomolecule and culturing the at least one droplet for sufficient time to perform a reaction.

A “droplet” as used herein comprises an droplet of aqueous fluid in oil, wherein said biomolecules or bioentities are provided in said droplets.

A “bioentity” as used herein may include but is not limited to, molecules, macromolecules, catalysts, viruses, prions, microbes, cells or cell fragments and organisms.

A “biomolecule” as used herein may refer to any type of target material, including but not limited to nucleic acids, polypeptides and peptides, ribonucleoproteins, a protein-nucleic acid complex, metabolites, beads, lipids, nanoparticles, liposomes, micelles, sugars, carbohydrates, glycoproteins, microbes, viruses or viral-like particles, cell secreting modification and/or an engineering reagents, polymers, polymersomes, molecular imprinted polymers, polymer complexes, dendrimer scaffolds, chromosomes, chromosome fragments, enzymes, chromosome/protein/chromatin complexes, aptamers, affimers and other non-antibody binding proteins/molecules, small molecules, therapeutics including membrane impermeable drugs, cryoprotectants, exogenous organelles, molecular probes, nanodevices, nanoparticles, organisms, genome editing reagents as defined herein and transfection or transduction reagents.

In one embodiment, the droplet is cultured for sufficient time for the biomolecule to react with the bioentity. As used herein “react” refers to any form of transformation or transfection or alteration of the bioentity by the biomolecule whether it is permanent or transient. The skilled person will appreciate that the culture time is specific to the type of reaction and the nature of the bioentities and biomolecules involved. However, examples of culture times may be at least 30 minutes, preferably at least one hour, more preferably at least 24 hours. In one example, culture times may be between 24 and 72 hours, more preferably between 48 and 72 hours, and even more preferably 48 hours.

In one embodiment, the method comprises providing at least two droplets, a first and a second droplet, wherein said first droplet comprises at least one bioentity, and said second droplet comprises at least one biomolecule, and wherein the bioentity and biomolecule are distributed between the at least two droplets such that the bioentity, and biomolecule are not all present simultaneously in a single droplet; wherein the method further comprises fusing said first and second droplets such that the bioentity and the biomolecule are present simultaneously in a fused droplet; and culturing the fused droplet.

In a further embodiment of the invention, there is provided a method of performing genome editing in a microfluidic droplet the method comprising providing at least one microfluidic droplet wherein said droplet comprises a cell or cell fragment, or nucleic acid derived therefrom, and genome editing reagents; and culturing the at least one droplet for sufficient time to perform genome editing in the cell or cell fragment.

As used herein, the term “genome editing” also means “gene editing” and such terms can be used interchangeably. Genome or gene editing refers to any modification or alteration, including the addition, deletion or substitution of at least one nucleotide in any region or part thereof of a target organism's genome. In other words, such an alteration may be in the coding or non-coding region of the genome. As such, “genome reagents” refers to the reagents necessary to carry out genome editing. Preferably, the genome editing reagents comprise a nuclease and a target DNA binding reagent.

Specifically, genome editing is a technique that uses targeted DNA double-strand breaks (DSBs) to stimulate genome editing through homologous recombination (HR)-mediated recombination events. To achieve effective genome editing via introduction of site-specific DNA DSBs, four major classes of customisable DNA binding proteins can be used: meganucleases derived from microbial mobile genetic elements, ZF nucleases based on eukaryotic transcription factors, transcription activator-like effectors (TALEs) from Xanthomonas bacteria, and the RNA-guided DNA endonuclease Cas9 from the type II bacterial adaptive immune system CRISPR (clustered regularly interspaced short palindromic repeats). Meganucleases, ZF, and TALE proteins all recognize specific DNA sequences through protein-DNA interactions. Although meganucleases integrate nuclease and DNA-binding domains, ZF and TALE proteins consist of individual modules targeting 3 or 1 nucleotides (nt) of DNA, respectively. ZFs and TALEs can be assembled in desired combinations and attached to the nuclease domain of FokI to direct nucleolytic activity toward specific genomic loci.

Upon delivery into host cells via the bacterial type III secretion system, TAL effectors enter the nucleus, bind to effector-specific sequences in host gene promoters and activate transcription. Their targeting specificity is determined by a central domain of tandem, 33-35 amino acid repeats. This is followed by a single truncated repeat of 20 amino acids. The majority of naturally occurring TAL effectors examined have between 12 and 27 full repeats.

These repeats only differ from each other by two adjacent amino acids, their repeat-variable di-residue (RVD). The RVD determines which single nucleotide the TAL effector will recognize: one RVD corresponds to one nucleotide, with the four most common RVDs each preferentially associating with one of the four bases. Naturally occurring recognition sites are uniformly preceded by a T that is required for TAL effector activity. TAL effectors can be fused to the catalytic domain of the FokI nuclease to create a TAL effector nuclease (TALEN) which makes targeted DNA double-strand breaks (DSBs) in vivo for genome editing. The use of this technology in genome editing is well described in the art, for example in U.S. Pat. Nos. 8,440,431, 8,440,432 and 8,450,471. Cermak T et al. describes a set of customized plasmids that can be used with the Golden Gate cloning method to assemble multiple DNA fragments. As described therein, the Golden Gate method uses Type IIS restriction endonucleases, which cleave outside their recognition sites to create unique 4 bp overhangs. Cloning is expedited by digesting and ligating in the same reaction mixture because correct assembly eliminates the enzyme recognition site. Assembly of a custom TALEN or TAL effector construct involves two steps: (i) assembly of repeat modules into intermediary arrays of 1-10 repeats and (ii) joining of the intermediary arrays into a backbone to make the final construct. Accordingly, using techniques known in the art it is possible to design a TAL effector that targets a desired nucleic acid sequence as described herein.

Another genome editing method that can be used according to the various implementations of the invention is CRISPR. The use of this technology in genome editing is also well described in the art, for example in U.S. Pat. No. 8,697,359 and references cited herein. In short, CRISPR is a microbial nuclease system involved in defence against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

One major advantage of the CRISPR-Cas9 system, as compared to conventional gene targeting and other programmable endonucleases, is the ease of multiplexing, where multiple genes can be mutated simultaneously simply by using multiple sgRNAs each targeting a different gene. In addition, where two sgRNAs are used flanking a genomic region, the intervening section can be deleted or inverted (Wiles et al., 2015).

Cas9 is thus the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. Accordingly, using techniques known in the art it is possible to design sgRNA molecules that targets a desired nucleic acid sequence as described herein.

In one embodiment, the method comprises providing at least two droplets, a first and a second droplet, wherein said first droplet comprises a cell or cell fragment, or nucleic acid derived therefrom, and said second droplet comprises genome editing reagents; wherein the genome editing reagents comprise at least one target DNA-binding reagent, at least one nuclease, and preferably a transfection or transduction reagent; wherein the cell or cell fragment, or nucleic acid derived therefrom, and genome editing reagents are distributed between the at least two droplets such that the cell or cell fragment, or nucleic acid derived therefrom, and genome editing reagents are not all present simultaneously in a single droplet; wherein the method further comprises fusing said first and second droplets such that the cell or cell fragment, or nucleic acid derived therefrom, and the genome editing reagents are present simultaneously in a fused droplet; and culturing the fused droplet.

By “target DNA-binding reagent” is meant any reagent that can bind at least one target nucleotide, preferably a target nucleotide sequence. In one embodiment, the target DNA-binding reagent comprises or consists of a sgRNA nucleic acid or a sgRNA molecule. In another embodiment, the target DNA-binding reagent may be a TAL-effector DNA binding domain. In a further alternative embodiment, the target-DNA binding reagent may be a zinc finger DNA-binding domain.

By “sgRNA” (single-guide RNA) is meant the combination of tracrRNA and crRNA in a single RNA nucleic acid or molecule, preferably also including a linker loop (that links the tracrRNA and crRNA into a single molecule).“sgRNA” may also be referred to as “gRNA” and in the present context, the terms are interchangeable. The sgRNA or gRNA provide both targeting specificity and scaffolding/binding ability for a CRISPR enzyme. A gRNA may refer to a dual RNA molecule comprising a crRNA molecule and a tracrRNA molecule.

Where the target DNA-binding reagent comprises a sgRNA nucleic acid, the reagent may be a nucleic acid construct comprising a sgRNA nucleic acid and a regulatory sequence operably linked to the sgRNA nucleic acid. The regulatory sequence may be any form or promoter, such as a constitutive, strong, regulated or inducible promoter that leads to expression of the sgRNA nucleic acid when expressed in the target cell. The same nucleic acid construct may also comprise a nuclease, as described below, which may be operably linked to the same or a different regulatory sequence.

By “TAL effector DNA binding domain” (transcription activator-like (TAL) effector) or TALE is meant a protein sequence that can bind the genomic DNA target sequence and that can be fused to the cleavage domain of an endonuclease such as FokI to create TAL effector nucleases or TALENS or meganucleases to create megaTALs. A TALE protein is composed of a central domain that is responsible for DNA binding, a nuclear-localisation signal and a domain that activates target gene transcription. The DNA-binding domain consists of monomers and each monomer can bind one nucleotide in the target nucleotide sequence. Monomers are tandem repeats of 33-35 amino acids, of which the two amino acids located at positions 12 and 13 are highly variable (repeat variable diresidue, RVD). It is the RVDs that are responsible for the recognition of a single specific nucleotide. HD targets cytosine; NI targets adenine, NG targets thymine and NN targets guanine (although NN can also bind to adenine with lower specificity).

By “zinc finger DNA binding domain” is meant a protein or polypeptide sequence comprising the zinc finger structural motif and that can bind to a specific sequence in double or single stranded DNA. The DNA binding domain of zinc fingers may contain between one and six, preferably between three and six individual zinc finger repeats that can each recognise between 9 and 18 base pairs.

By “nuclease” is meant any enzyme that comprises a DNA cleavage domain. In other words an enzyme that can cleave at least one DNA strand. In one embodiment, the nuclease is an endonuclease, more preferably a restriction endonuclease such as FokI. In other embodiments, the nuclease is a CRISPR enzyme. The nuclease may be a polypeptide or a nucleic acid that encodes for the nuclease. If the latter, by “nuclease” may also be meant a nucleic acid construct comprising a nucleic acid encoding for a nuclease, wherein preferably the nucleic acid is operably linked to a regulatory sequence. A regulatory sequence is described above.

By “CRISPR enzyme” is meant an RNA-guided DNA endonuclease that can associate with the CRISPR system. Specifically, such an enzyme binds to the tracrRNA sequence. In one embodiment, the CRIPSR enzyme is a Cas protein (“CRISPR associated protein), preferably Cas9 or a Cpf protein, such as Cpf1. The Cas9 protein may also be modified to improve activity. For example, the Cas9 protein may comprise the D10A amino acid substitution, this nickase cleaves only the DNA strand that is complementary to and recognized by the gRNA. In an alternative embodiment, the Cas9 protein may alternatively or additionally comprise the H840A amino acid substitution, this nickase cleaves only the DNA strand that does not interact with the sRNA. In this embodiment, Cas9 may be used with a pair (i.e. two) sgRNA molecules (or a construct expressing such a pair) and as a result can cleave the target region on the opposite DNA strand, with the possibility of improving specificity by 100-1500 fold. In a further embodiment, the Cas9 protein may comprise a D1135E substitution. The Cas9 protein may also be the VQR variant. Alternatively, the Cas protein may comprise a mutation in both nuclease domains, HNH and RuvC-like and therefore is catalytically inactive. Rather than cleaving the target strand, this catalytically inactive Cas protein can be used to prevent the transcription elongation process, leading to a loss of function of incompletely translated proteins when co-expressed with a sgRNA molecule. An example of a catalytically inactive protein is dead Cas9 (dCas9) caused by a point mutation in RuvC and/or the HNH nuclease domains (Komor et al., 2016 and Nishida et al., 2016).

The Cas protein, such as Cas9, may also be further fused with a repression effector, such as a histone-modifying/DNA methylation enzyme or a Cytidine deaminase (Komor et al.2016) to effect site-directed mutagenesis. In the latter, the cytidine deaminase enzyme does not induce dsDNA breaks, but mediates the conversion of cytidine to uridine, thereby effecting a C to T (or G to A) substitution.

Cas9 expression plasmids for use in the methods described herein can be constructed as described in the art.

In an alternative embodiment, the CRISPR enzyme is a MADzyme, such as MAD7 (nuclease from the Eubacterium rectale genome (WP_055225123.1)).

In a further embodiment, the CRISPR enzyme is tagged with a suitable marker that allows sorting of the droplets based on the presence of the marker. For example, the marker may be a fluorescent tag. In one embodiment, the CRISPR enzyme may be tagged with GFP. As described herein the method may comprise the step of analysing the droplet for a predetermined property and sorting the droplet on the basis of the analysis. In one embodiment, the predetermined property may be the presence of the marker. For example, the expression of GFP. The presence of GFP can be used to determine if the droplet comprises genome editing reagents.

In one embodiment, where the target DNA-binding reagent and/or nuclease is a nucleic acid construct, the construct may be integrated in a stable form in the genome of the target cell or cell fragment or any nucleic acid derived therefrom. In an alternative embodiment, the nucleic acid construct or constructs are not integrated (i.e. are transiently expressed). As such, the methods described herein may be used to cause stable or transient modifications, as described above to a target genome.

As used herein, a “transfection or transduction reagent” may refer to any agent or reagent that is capable of inducing transformation or transduction of a biological entity such as a cell. Alternatively, when used in the context of a cell transfection or transduction, such a reagent may be defined as any reagent capable of causing membrane disruption and consequently intracellular delivery of a biomolecule into the cell.

The terms “introduction”, “transfection” or “transformation” can be used interchangeably and encompass the transfer of an exogenous biomolecule, such as a polynucleotide into a host cell, irrespective of the method used for transfer. Examples of suitable transformation reagents may include any biochemical means, such as but not limited to, chemicals that increase free DNA uptake, detergents, pore-forming agents such as Lipofection or Lipofectamine, non-liposomal lipids such as Fugene, activated dendrimers such as PolyFect, cationic polymers such as TurboFect, lipopolyplexes such as TransIT, ligand conjugates, cell ghosts, cell penetrating peptides, exosomes, DNA or RNA-coated particle bombardment and vesicles. Other examples use nanotechnology, such as but not limited to nanotubes and nanodevices, lipid nanocarriers, inorganic nanocarriers and polymer nanocarriers.

The term “transduction” encompasses the transfer of an exogenous biomolecule, such as a polynucleotide into a host cell, using a virus or viral vector. Accordingly, in one embodiment, the transduction reagent may be a virus or viral vector.

The term “nucleic acid construct” or “vector” (such terms may be used interchangeably) may be used herein to refer to a nucleic acid molecule capable of transferring or transporting another nucleic acid molecule. A vector may include sequences that direct autonomous replication in a cell, or may include sequences sufficient to allow integration into host cell DNA. Useful vectors include, but are not limited to, plasmids (such as DNA plasmids or RNA plasmids), liposomes, episomes, transposons, cosmids, bacterial artificial chromosomes viral vectors and synthetic vectors or carriers. Synthetic carriers may be made from at least one of lipids, polymers and inorganic nanomaterials.

The term viral vector may refer either to a virus or viral particle capable of transferring a nucleic acid into a cell. Viral vectors contain structural and/or functional genetic elements that are primarily derived from a virus. Typically viral vectors exploit the viral infection pathway to enter cells but avoid the subsequent expression of viral genes that leads to replication and pathogenicity. This is achieved by deleting coding regions of the viral genome and replacing them with the DNA to be delivered, which either integrates into host chromosomal DNA or exists as an episomal vector (Stewart et al. 2006). Examples of useful viral vectors include replication defective retroviruses and lentiviruses. In one embodiment, the vector is a viral vector and comprises at least one target DNA-binding reagent, such as a sgRNA nucleic acid and a nuclease, such as Cas9. In one particular embodiment, the Cas9 may be GFP-Cas9. Preferably the viral vector also comprises at least one regulatory sequence operably linked to the sgRNA and/or the Cas9 enzyme.

Use of a transfection or transduction agent in this system is preferable but not essential because a physical or mechanical mechanism for transfection or transduction could alternatively be employed.

Accordingly, in some embodiments of the invention, the method may further comprise the step of transfecting a biomolecule, such as a genome editing reagent into a bioentity such as a cell, wherein preferably transfection or transduction is by membrane-disruption, preferably by any physical or mechanical means. In this example, physical or mechanical means include but are not limited to, electroporation, nanoneedles, injection of the DNA directly into the cell (microinjection), gene guns (or biolistic particle delivery systems (biolistics)), ultrasound-mediated gene transfection, optical or laser transfection, including optoporation, photoporation, laserfection and laser-induced convective transmembrane transport and transfection using silicon carbide fibres, thermal transfection and using mechanical fluid shear, squeezing or cavitation or osmotic or hydrostatic forces. As discussed below, the method may also comprise the step of determining that a cell has been transfected, and preferably selecting transfected cells. As discussed above, in one example this may be achieved using a viral vector expressing both the target DNA binding reagents and a tagged, for example fluorescently-tagged nuclease, such as GFP-Cas9.

In another embodiment of the invention, the method may comprise providing at least a first, second and third droplet, wherein the first droplet comprises a cell or cell fragment, or nucleic acid derived therefrom, the second droplet comprises a target DNA-binding reagent and/or a nuclease and the third droplet comprises a nuclease and/or a transfection or transduction reagent, wherein the method comprises fusing the first, second and third droplets, and culturing the fused droplet.

In a further embodiment of the invention, the method may comprise providing at least a first, second, third and fourth droplet, wherein the first droplet comprises at least one cell or cell fragment, or nucleic acid derived therefrom, the second droplet comprises at least one target DNA-binding reagent, the third droplet comprises at least one nuclease and the fourth droplet comprises a transfection or transduction reagent, wherein the method comprises fusing the first, second, third and fourth droplets, and culturing the fused droplet.

In one embodiment, the droplet or the first droplet—i.e. the droplets comprising at least one cell or cell fragment additionally comprises cell media (also known as “growth medium” or “culture medium”) The skilled person will appreciate that the cell media will be any media capable of supporting the growth of the cell(s) and may be specific to the cell type and also the reaction to be performed. For example, the cell media may include serum or be serum-free.

In a further embodiment, the method comprises preparing the at least one microfluidic droplet from at least one reservoir, as described in further detail below.

In one embodiment, the droplet may be provided to an incubator and cultured for sufficient time for genome editing to be performed in the cell, cell fragment or on any nucleic acid molecule derived therefrom. In another additional or alternative embodiment, the droplet is cultured for sufficient time for cell division to occur. In one embodiment, the culture time may be at least 30 minutes, preferably at least one hour, more preferably at least 24 hours. In one example, the culture time may be between 24 and 72 hours, more preferably between 48 and 72 hours.

By “fusion” is meant any form of coalescence of one or more droplets. In one embodiment, fusion may be passive fusion. That is, by a mechanism that does not require active control or electricity. One advantage of passive droplet fusion techniques is that the possibility of inter-droplet contamination is lower, while one disadvantage is that the rate of fusion is generally slower than using active fusion (Simon & Lee, 2012). In one embodiment, passive fusion may be performed by altering surfactant concentration, altering droplet viscosity, altering droplet surface tension, reducing or draining the volume of oil between droplets or by physical constriction or physical collision. In one example, physical collision may be achieved using a widened and/or gradually tapering channel or expansion volume. This feature removes the spacing between droplets, subsequently permitting contact and ultimately fusion between adjacent droplets.

In another embodiment, fusion may be active fusion. In one embodiment, active fusion may be performed using electrocoalescence, dielectrophoresis, by electrically charging at least one droplet for fusing by electrostatic attraction, optical tweezers, electrowetting, lasers, acoustics, thermal energy or physical forces.

In a further embodiment, the method further comprises determining whether the droplet or first droplet comprises no bioentities, one bioentity or a plurality of bioentities. More preferably, the method comprises determining whether the droplet or first droplet comprises no cells, one cell or a plurality of cells and sorting the droplet on the basis of the determination, wherein droplets with no or a plurality of cells are preferably passed to a waste outlet. Preferably, the step of determining and sorting droplets containing only one bioentity is performed before the droplet is cultured. More preferably, the step of determining whether the droplet contains a single entity is performed prior to any fusion, such that fusion is performed only between a droplet containing single entity, such as cell, a single cell fragment of a single nucleic acid and a droplet containing a biomolecule, such as genome editing reagents. However, the skilled person will appreciate that the methods may also be used to combine multiple entities and biomolecules.

It will be understood that a droplet may be sorted using one of at least one of a variety of techniques. Such techniques include, but are not limited to, dielectrophoresis, magnetophoresis, electro-osmosis, and the like.

In another embodiment of the invention, the method further comprises analysing the droplet or fused droplet for at least one predetermined property, and preferably sorting the droplet on the basis of the analysis. For example, the phenotype of the droplet may be analysed.

As used herein, a “predetermined property” or “phenotype” of the droplet may be used to refer to determining whether the droplet has one, none or multiple biological entities, such as cells in the droplet. Alternatively, as described above, the property or phenotype may refer to determining whether the droplet expresses a marker indicative of transfection of one or more genome editing reagents (a transfection control). As discussed above, in one example, a CRISPR enzyme may be tagged, for example fluorescently, and in this context, determining a property or phenotype would refer to determining fluorescence as indicative of expression of the fluorescent-tagged CRISPR enzyme. This analysis may be particularly useful where the method and system allow for the formation of a single droplet from at least one inlet that comprises a plurality of biological entities, such as cell and genome editing reagents.

In a further alternative, the property or phenotype may be indicative of whether there has been a reaction between the biological entity and the biomolecule, for example, whether the target gene has been mutated with the genome editing reagents. The skilled person would be aware of a number of genome editing and analysis tools that could be used for this purpose in the microfluidic droplets.

In one embodiment, analysis of the droplet comprises measuring a phenotypic change caused by genome editing. Preferably, such phenotypic change(s) can be measured using fluorescent assays. In one example, fluorescent assays can be used to detect:
1. changes in cell signaling pathways as measured by ion fluxes;
2. transcription of a fluorescent reporter gene eg GFP;
3. production of a specific protein using two hybrid assays;
4. secretion of a specific protein (eg antibody or enzyme or Cytokine or growth factor) or biomarker;
5. production of a specific cell surface protein;
6. expression of a specific mRNA; and/or
7. presence of a specific nucleic acid sequence by PCR or amplification technique.

Preferably, analysis of the droplet or the fused droplet is performed following culturing of the droplet.

In a further embodiment of the invention, the method further comprises splitting the droplet or the fused droplet into a least a first and second daughter droplet, although the droplet may be split into multiple daughter droplets. Preferably, splitting the droplet or fused droplet is performed following culturing of the droplet or fused droplet.

More preferably each daughter droplet contains at least one entity, such as one cell, cell fragment of nucleic acid derived therefrom, and therefore the number of daughter droplets may depend on the extent of cell division that has occurred during culturing of the droplet. Accordingly, the method further comprises analysing and sorting the daughter droplets for the presence of one entity, such as a cell. Again, daughter droplets containing no or a plurality of entities, such as cells, may be passed to a waste outlet.

In a further embodiment, the at least one daughter droplet may be analysed and sorted into one of a dispenser, a further analytical device and a waste outlet.

In some preferred embodiments of the method, providing the droplet comprises providing a plurality of droplets, wherein a droplet of the plurality of droplets is separated from a neighbouring droplet of the plurality of droplets by a spacing fluid. Once the droplets are split, a said first droplet may then be separated from a neighbouring said first droplet by a second spacing fluid, and a said second droplet may be separated from a neighbouring said second droplet by a third spacing fluid.

It will be appreciated that the first, second and third spacing fluids may in fact be the same, single spacing fluid. Alternatively, the first, second and third spacing fluids may be different spacing fluids. The type of spacing fluid used for (parent) droplets, first droplets and second droplets may be chosen dependent on the properties of the droplets and/or the type(s) of analysis performed on the droplets since a spacing fluid may impede on a particular type of analysis of the droplets. In some embodiments, the first and/or second spacing fluids may be removed before analysing the first droplets and/or before sorting or analysing the second droplets.

In some preferred embodiments of the method, the first, second and third spacing fluids may be oils and/or water-in-oil emulsions.

In a particularly preferred embodiment, the method may comprise providing at least one microfluidic droplet, wherein said droplet comprises a cell or cell fragment and genome editing reagents; transfecting the genome editing reagents into said cell, wherein preferably transfection is by membrane-disruption, selected from physical, mechanical, electrical, thermal and optical techniques; preferably determining whether the droplet comprises no cells, one cell or a plurality of cells and sorting the droplet on the basis of the determination, wherein droplets with no or a plurality of cells are preferably passed to a waste outlet; culturing the droplet, preferably for sufficient time to allow cell division; and analysing the droplets for a predetermined property or phenotype, wherein preferably the analysis comprises determining whether the cell or cell fragment has been transfected with the genome editing reagents as described above. The method may further comprise splitting said droplet into at least a first and second daughter droplet and/or dispensing the droplet. As discussed above, in one embodiment, it may be possible to determine if the cells have been successfully transfected with the genome editing reagents if the genome editing reagents are tagged, such as with a fluorescence marker. Accordingly, at this analysis stage fluorescence of the expressed genome editing reagents would be indicative of successful transfection.

In another implementation there is provided a microfluidic system for reacting a least one biomolecule with at least one single entity, the system comprising or consisting of at least one reservoir, wherein the at least one reservoir comprises a plurality of biological entities and biomolecules; and an oil reservoir, a droplet formation device for preparing at least one droplet from the at least one reservoir; and an incubator for culturing the droplet for sufficient time to perform a reaction between the biological entity and the biomolecule.

In one embodiment, there is provided a microfluidic system for performing genome editing in a microfluidic droplet, the system comprising or consisting of at least one reservoir, wherein the at least one reservoir comprises a plurality of biological entities and biomolecules; and an oil reservoir, a droplet formation device for preparing at least one droplet from the at least one reservoir; and an incubator for culturing the droplet for sufficient time to perform genome editing.

In one embodiment, the system comprises a plurality of reservoirs, wherein each reservoir comprises a plurality of bioentities, such as cells, cell fragments or nucleic acid as well as at least one, but preferably a plurality of biomolecules, such as genome editing reagents. In this embodiment, the bioentities of one reservoir may be different from the bioentities of the at least one other reservoir, such that when the droplets are formed using the droplet generation device, the droplets contain a different combination of bioentities and biomolecules. For example, the bioentity may be a cell or cell fragment or nucleic acid derived therefrom and reservoir may contain cells, cell fragments or nucleic acid from a different cell type or alternatively, the same cell type but from a different sample source. In a further embodiment, the reservoir comprising cells or cell fragments may additionally comprise growth medium. The skilled person will appreciate that the growth medium may vary depending on cell type.

In an alternative embodiment, the system comprises at least two reservoirs, wherein the first reservoir comprises a plurality of biomolecules, such as cells, cell fragments or nucleic acid and optionally a growth medium, and the second reservoir contains a plurality of biomolecules, such as genome editing reagents. In this embodiment, the droplet generation device prepares at least one droplet from the first and the at least second reservoir. As a result, multiple droplets each comprising genome editing reagents and cells, cell fragments or nucleic acid from a single cell type or sample source may be generated. In a further embodiment, the system may comprise at least three reservoirs, wherein the first reservoir comprises cells, cell fragments or nucleic acid and optionally a growth medium, the second reservoir comprises genome editing reagents and the third reservoir comprises transfection or transduction reagents, as described herein. Again, in this embodiment, the droplet generation device prepares at least one droplet from the first, second and third reservoir. However, this embodiment may not be necessary if the system alternatively comprises a transfection or transduction device.

Accordingly, in an alternative embodiment, the system comprises a transfection or transduction device configured to disrupt the membrane of a cell by, for example, physical, mechanical, electrical, thermal or optical means, as described herein.

In a further implementation, there is provided a microfluidic system for performing genome editing in a microfluidic droplet, the system comprising a first reservoir, wherein the first reservoir comprises a plurality of bioentities, such as cells or cell fragments; a second reservoir comprising a plurality of biomolecules, such as genome editing reagents; at least one oil reservoir; a first droplet formation device for preparing at least one droplet from the first reservoir and the oil reservoir; a second droplet formation device for preparing at least one droplet from the second reservoir and oil reservoir; a droplet fusion region for fusing the at least one droplet prepared from the first and second droplet generation device; and an incubator for culturing the droplet for sufficient time to perform genome editing.

In one embodiment, the first and second droplet formation device may be the same device, and can alternate between forming a droplet from the first and second reservoir respectively. In an alternative embodiment, the first and second droplet formation devices are separate devices.

In one embodiment, the system comprises one oil reservoir that can be used to form droplets by the first and second droplet generation device. In an alternative embodiment, the system comprises a first and second oil reservoir wherein the first droplet generation device can prepare a droplet from the first reservoir and the first oil reservoir and the second droplet generation device can prepare a droplet from the second droplet generation device and the second oil reservoir.

In a further embodiment, the system may further comprise a third reservoir, wherein the first reservoir comprises a plurality of cells, cell fragments of nucleic acid, the second reservoir comprises genome editing reagents and the third reservoir comprises a nuclease and/or transfection or transduction reagents. In this embodiment, the droplet generation device generates a droplet from the first, second and third reservoir and at least one oil reservoir. In one embodiment, the system may comprise a single oil reservoir that feeds into the first, second and third oil reservoirs. Alternatively, the system may comprise a first, second and third oil reservoir wherein the first, second and third droplet formation device forms a droplet from the first, second and third reservoir and the first, second and third oil reservoir respectively.

In one embodiment, the system may comprise a droplet fusion region for fusing a plurality of droplets formed from the plurality of droplet formation devices. Accordingly, in one embodiment, the droplet fusion region allows the fusion of a droplet formed from the first droplet formation device with a droplet formed by the second and/or third droplet formation device. In one example, this allows the fusion of a droplet comprising cells, cell fragments or nucleic acid with a droplet comprising genome editing reagents and/or nucleases and/or transfection or transduction reagents. As discussed above, fusion may be by passive or active means.

Accordingly, in one embodiment, the droplet fusion region fuses a plurality of droplets by passive fusion. In one example, the droplet fusion region may comprise a widened and/or gradually tapered channel that results in an expansion volume. This feature removes the spacing between droplets, subsequently permitting contact and ultimately fusion between adjacent droplets. Accordingly, fusion is achieved using physical constriction or collision. Alternatively, active fusion can be performed by reducing the surfactant concentration or removing the surfactant.

In another embodiment, the droplet fusion region fuses a plurality of droplets by active fusion. Accordingly, in one embodiment, the droplet fusion region may comprise an electric field generator for generating an electric field for fusing the droplets by electrocoalescence. Alternatively or in addition, the droplet fusion region may comprise one or more charging devices for electrically charging droplets for fusion by electrostatic attraction. Alternatively, the droplet fusion region may comprise laser, acoustic or heat activated fusion. In a further alternative, fusion might be activated by physicochemical means, such as by surfactant/interface properties and compositions.

In one embodiment, the droplet fusion region is placed before the incubator in a fluidic flow path direction in the microfluidic system, as this ensures that droplets comprising all the reagents necessary for a reaction, such as genome editing, are contained within a single droplet.

In one embodiment, the system comprises at least one analyser and at least one droplet sorting region for sorting a droplet based on one or more predetermined properties.

In a further embodiment, the system comprises a first analyser configured to determine whether the droplet comprises a single entity such as a single cell, cell fragment or nucleic acid, no entities, such as no cells, or a plurality of entities, such as a plurality of cells. Preferably, the first analyser is placed before the incubator in a fluidic flow in the microfluidic device. More preferably, the first analyser is placed before the droplet fusion region in a fluidic flow path of the microfluidic device. In a further embodiment, the system further comprises a first droplet sorting region, which sorts the droplet based on the determination of the analysis. Preferably, droplets comprising no or a plurality of entities are sorted by the droplet sorting region and passed to a second fluidic flow path of the system and preferably passed to a waste outlet. This ensures that only droplets comprising single entities such as single cells continue through the microfluidic device and are fused and/or incubated.

In another embodiment, the system comprises a second analyser configured to analyse the content of the droplet and a second droplet sorting region for sorting the droplet on the basis of the analysis. For example, the analyser may comprise a fluorescence detector. Preferably, the second analyser and second droplet sorting region is placed after the incubator such that the droplets can be analysed after sufficient time has passed for a reaction to occur in the droplets.

It will be understood that a droplet may be sorted using one of a variety of techniques. Such techniques include, but are not limited to, dielectrophoresis, magnetophoresis, electro-osmosis, and the like. The skilled person will appreciate that depending on the identified constituents, some techniques for sorting the droplet may be preferred over others in order to minimise or prevent a negative impact on the droplet during the sorting process.

In one embodiment, the incubator is configured to hold the droplets at a controlled temperature and for sufficient time for a reaction to be performed. In one example, in use, the droplets are held in the incubator for sufficient time for genome editing to be performed on the target bioentity and/or for at least one round of cell division to be performed. In one embodiment, the incubator is placed after the first analyser and in front of the second analyser in a fluidic flow path in the microfluidic system as this ensures that only droplets determined to contain single entities are incubated or cultured and subsequently analysed.

In a further embodiment, the microfluidic system further comprises a droplet splitting region for splitting a droplet into at least one daughter droplet. Preferably, the droplet splitting region is placed downstream of the incubator in a fluidic flow in the microfluidic device. More preferably, the droplet splitting region is placed downstream of the second analyser and second droplet sorting region. In one embodiment, droplets identified by the second analyser to have a desired characteristic or predetermined property are passed to the droplet splitting region. For example, droplets that are determined by the analyser to have multiple cells as a result of cell division in the incubation step may be passed to the droplet splitting region. In a further embodiment, droplets determined by the second analyser not to have the desired characteristic or predetermined property may be sorted to a second fluidic flow that leads to a waste outlet.

It will be appreciated that a variety of techniques may be used to split the droplet into first and second droplets. These techniques include, but are not limited to, geometry-mediated splitting, or droplet splitting using electric field, heat or lasers. It will be understood that one technique may be preferable over another dependent on the type of droplet(s) to be split.

In a yet further embodiment, the microfluidic system further comprises at least one dispensing unit for dispensing a droplet. In one embodiment, droplets analysed by the second analyser to have a desired phenotype or a predetermined property may be sorted directly to a dispensing unit for storage and/or subsequent analysis. In an alternative or additional embodiment, the microfluidic system may comprise a dispensing unit placed downstream of the droplet splitting region. In either embodiment, the droplet may be dispensed into, for example, individual wells of a microtiter plate where the one or more entities contained in the single droplet (or multiple droplets) may be stored and afterwards retrieved for further processing or analysis, or for subsequent use.

In a further preferred embodiment of the microfluidic system, the dispensing unit comprises a reservoir for storing a growth media fluid wherein the dispensing unit is configured to dispense a said split droplet in the growth media fluid. This may be particularly preferable, as the dispensing step and the step of placing the single droplet containing the one or more entities in a growth media fluid may be combined in a single step. A droplet may be prepared from the growth media fluid which contains the entity or entities-containing droplet, which may then be dispensed into, for example individual wells of a microtiter plate.

In another aspect, there is provided a microfluidic device comprising at least one sample input channel for receiving a fluid comprising a plurality of biological entities and biomolecules as described above, an oil input channel for receiving an oil, wherein the at least one sample and oil channels are fluidly connected to a droplet generating region for generating microfluidic droplets comprising at least one biomolecule and at least one biological entity, an incubator for culturing the droplet; and at least one output channel.

In one embodiment, there is provided a microfluidic product comprising a substrate comprising a first input channel for receiving a fluid comprising a plurality of cell or cell fragments or nucleic acid molecules and optionally genome editing reagents, a second input channel for receiving an oil, wherein the first and second input channels are fluidly connected to a first droplet generating region for generating microfluidic droplets comprising at least one cell or cell fragment, an incubator for culturing the droplet and at least one output channel.

In a further embodiment, the product comprises a first inlet channel for receiving a fluid comprising a plurality of cell or cell fragments, a second input channel for receiving an oil and a third inlet channel for receiving genome editing reagents. In this embodiment, the third input channel may be fluidly connected to a second droplet generating region.

Furthermore, in this embodiment, the microfluidic product further comprises a droplet fusion region for fusing at least one droplet prepared from the first droplet generation region with at least one droplet prepared from the second droplet generation region.

In a further embodiment, the product further comprises a first analyser and a single cell sorting region, wherein preferably said single cell sorting region is downstream of the droplet generating region(s).

In another embodiment, the product further comprises a second analyser and a sorting region where the droplet is analysed for a predetermined property and wherein the droplet is sorted dependent on the analysis. Preferably, the sorting region is downstream of the incubator.

In a further embodiment, the product further comprises a droplet splitting region connected to the at least one output channel.

In a typical embodiment, the input channels, droplet generating regions, incubator, droplet fusion region, single cell sorting region, sorting region, droplet splitting regions and at least one outlet channel are incorporated on a single substrate. The substrate may have the form of a flat plate bearing microfluidic channels fluidly connecting the incubator and above regions. It is preferably substantially optically transparent, and may be fabricated from a range of plastic materials, such as fluorinated or non-fluorinated plastics, glass, silicon or synthetic polymers. In a further embodiment, the polymer is selected from the group comprising polydimethylsiloxane, polyurethane and styrene-ethylene-butadiene-styrene. The single substrate may then be mounted vertically or at a suitable angle in the system/instrument with the output channel directed downwards towards a multi-well or microtiter plate. The instrument may then move the substrate to direct the output channel into a selected well.

In preferred embodiments the product is provided with a plurality of fluidic connections, which may be made automatically when the cartridge is inserted into the instrument. Generation of the emulsion may be performed on-cartridge or off-cartridge. For example, in embodiments the cartridge may be provided with reservoirs along one edge (so that these are in the correct orientation when the cartridge is vertical), to hold oil and aqueous medium (such as water and growth medium) for droplet-on-chip droplet generation.

The oil composition may comprise, e.g., fluorous and/or mineral oil, and, e.g., 25% vol/vol surfactant.

Preferably a surfactant is used to stabilise the aqueous microdroplet in the oil composition. The surfactant may comprise one or more surfactants, and may be a polymeric or a small molecule surfactant. Moreover, the surfactant may ionise relatively inefficiently (for example compared to the analyte). Such surfactants may have relatively poor surfactant properties, e.g., may be less good at preventing fusion of microdroplets, compared to other surfactants that are less suitable for mass spectrometry. For example, surfactants in an embodiment may comprise small molecules (e.g., having a molecular weight of less than 800 g/mol, more preferably less than 600 g/mol or 400 g/mol, e.g., 364 g/mol) and hence may be volatile. This may be advantageous for evaporation of the spray droplets allowing more charged analyte molecules to be in the gas phase for detection by the mass spectrometer.

In a further embodiment, the methods or techniques described herein can be implemented in a system of the type described in WO2016/193758, which is incorporated by reference.

In another implementation there is provided a microfluidic droplet for performing genome editing, the droplet comprising at least one cell or cell fragment, cell culture medium and genome editing reagents. In particular, we have identified that droplets of a size in the range of between 100 and 10,000 pL are capable of remaining viable for sufficient time for genome editing to occur in the encapsulated cell or cell fragment.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

### EXAMPLES

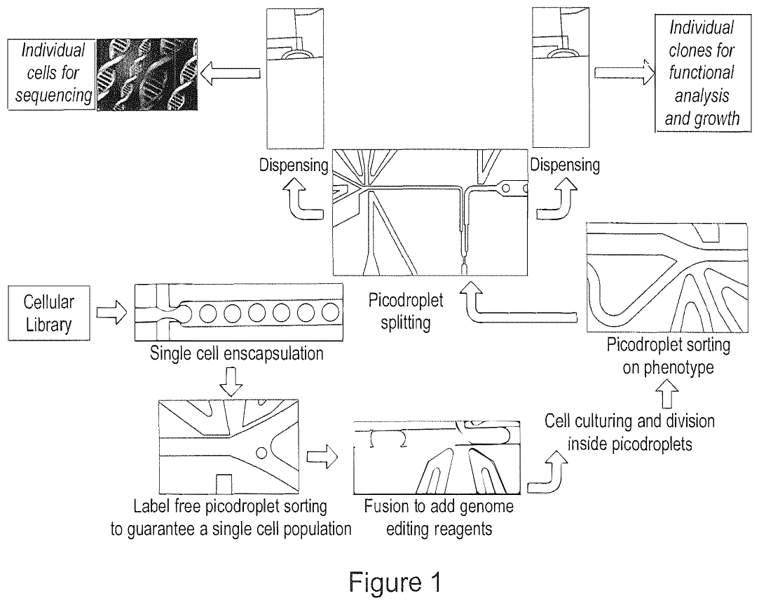

FIG. 1 shows a schematic illustration of one example of a genomic editing microfluidic workflow.

In this example, a plurality of cells is provided in a fluid, optionally including a growth medium. In the first step, individual droplets are formed from the fluid. As outlined above, this may be achieved by using, for example, T-junctions, Y-junctions, flow focusing devices, or other devices. The droplets which have been generated are, in this example, transported in a fluid of oil.

The individual droplets, which may or may not contain one or more cells, are then guided through the microfluidic device in an oil emulsion.

In a second optional step, the droplets in the oil emulsion, are guided towards a first analyser and single cell sorting region. Whether or not a single droplet contains one or more cells may be detected in the analyser, based on one or more of electrical, optical, thermal, acoustic, mechanical, temporal, spatial, and other physical characteristics of the droplet. Based on the analysis in the analyser, i.e. the determination as to whether a single droplet contains no, one or a plurality of target cells, the droplet may be sorted in the droplet sorting device. In this example, droplets that do not contain one or more cells or droplets that contain a plurality of cells are put to waste. Droplets that contain a single cell are guided towards a droplet fusion region.

In the next step, droplets containing a single cell are fused with one or more droplets containing genome editing reagents in a droplet fusion region. This droplet fusion region may in one embodiment comprise a widened and/or gradually tapered channel that results in an expansion volume. This feature removes the spacing between droplets, subsequently permitting contact and ultimately fusion between adjacent droplets. Accordingly, fusion is achieved using physical constriction or collision. In another embodiment, the droplet fusion region fuses a plurality of droplets by active fusion. Accordingly, in one embodiment, the droplet fusion region may comprise an electric field generator for generating an electric field for fusing the droplets by electrocoalescence. Alternatively or in addition, the droplet fusion region may comprise one or more charging devices for electrically charging droplets for fusion by electrostatic attraction. Alternatively, the droplet fusion region may comprise a laser, acoustic or heat activated fusion.

Each droplet containing single cells may be fused with at least one further droplets comprising genome editing reagents and optionally transfection or transduction reagents. Alternatively, each droplet containing single cells may be fused with at least two further droplets, one containing genome editing reagents and the second comprising transfection or transduction reagents. This latter alternative may not be necessary if the microfluidic system comprises a transfection or transduction device as described above.

As shown in FIG. 1, the droplet fusion region may be placed after the first analyser and single cell sorting region. Such a configuration ensures that droplets comprising only single cells are fused with the genome editing reagents and/or transfection or transduction reagents. This is important as if the droplets comprised multiple cells, the amount of genome editing reagents and/or or transfection or transduction reagents would have to be modified accordingly. Of course, conversely, if the droplets contained no cells there would be no point fusing such a droplet with genome editing reagents and/or or transfection or transduction reagents.

In an alternative embodiment, genome editing reagents may be added into the droplets containing the single cells as they flow through the microfluidic device. This may be achieved by using at least one, preferably a plurality of narrow hydrophilic channels to introduce the genome editing reagents into the droplets. The advantage of this technique is that is avoids having to prepare multiple droplet streams and fusing these streams.

Droplets which have been prepared from the fluid containing cells, genome editing reagents and optionally transfection and transduction reagents are then guided into an incubator. The incubator may be used to culture the droplets for sufficient time and at a suitably controlled temperature for genome editing to be performed in the cell. Additionally or alternatively, the incubator may be used to culture the droplets for sufficient time and a suitably controlled temperature for at least one round of cell division to occur.

In the next step, the incubated cells are passed to a second analyser and single cell sorting region. In this step, the content of the cells are determined. For example, the cells are analysed to determine if the target nucleic acid sequence has been mutated by the genome editing reagents. Alternatively or in addition, the cells are analysed to determine if one or more rounds of cell division has occurred. This will be clear from the presence of a plurality of cells in the droplet.

Again, as shown in FIG. 1, the second analyser and single cell sorting region is placed behind the incubator. This is to ensure that the second sorting step is not performed until the genome editing reaction is complete and/or suitable time has passed for cell division to occur, and is therefore necessary to be able to perform any meaningful downstream analysis.

In a next optional step, the analysed droplets are guided to a droplet splitting region. Preferably droplets comprising a plurality of cells are guided to the droplet splitting region where the droplets are split into a plurality of daughter droplets.

In a further optional step, the split droplets are guided to at least one, preferably a plurality of dispensing units for subsequent analysis, such as genome sequencing. Alternatively, the droplets may be dispensed into a further incubator to further grow the mutated cells and analyse their phenotype.

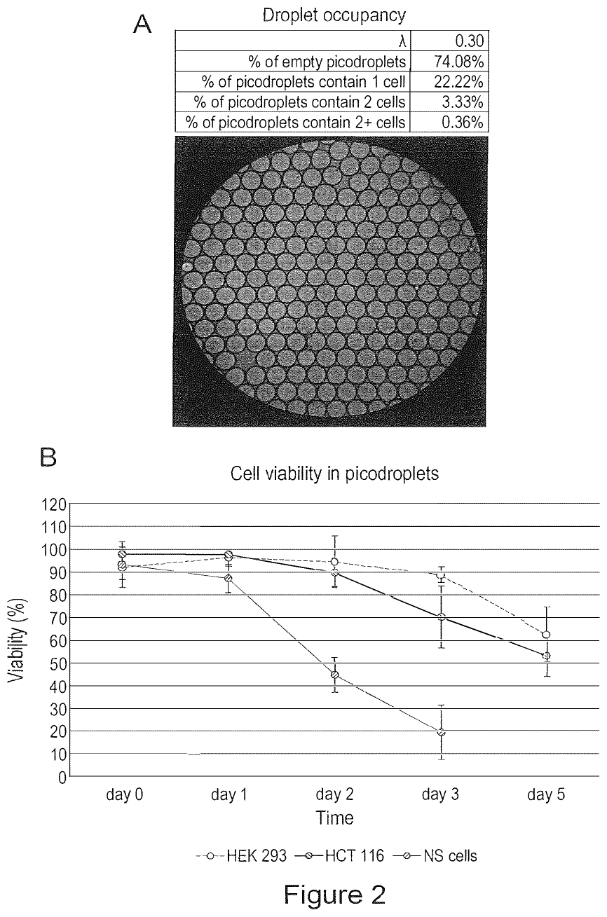

FIG. 2 shows the encapsulation of different types of adherent cells in picodroplets and quantification of their subsequent survival in picodroplets for prolonged period of time (5 days). In FIG. 2a HCT116 cells (a human colon cancer cell line) (1×106 cell/ml) were encapsulated in 300 pL picodroplets (bottom image). Droplets occupancy calculated for 1×10⁶ cell/ml is shown in top table. In FIG. 2b, HCT116, HEK-293 or mouse NS cells (1×10⁶ cell/ml) were encapsulated in 300 pL picodroplets for 5 days. Cells viability was tested every 24 hours immediately following cells retrieval from picodroplets. Viability was measured by NucleoCounter NC-3000 (ChemoMetec) using Acridine Orange/DAPI dye to label live/dead cells, respectively. Two measurement were taken in every time point/experiment. Graph represent mean−/+SD of 5 individual experiment (n=5).

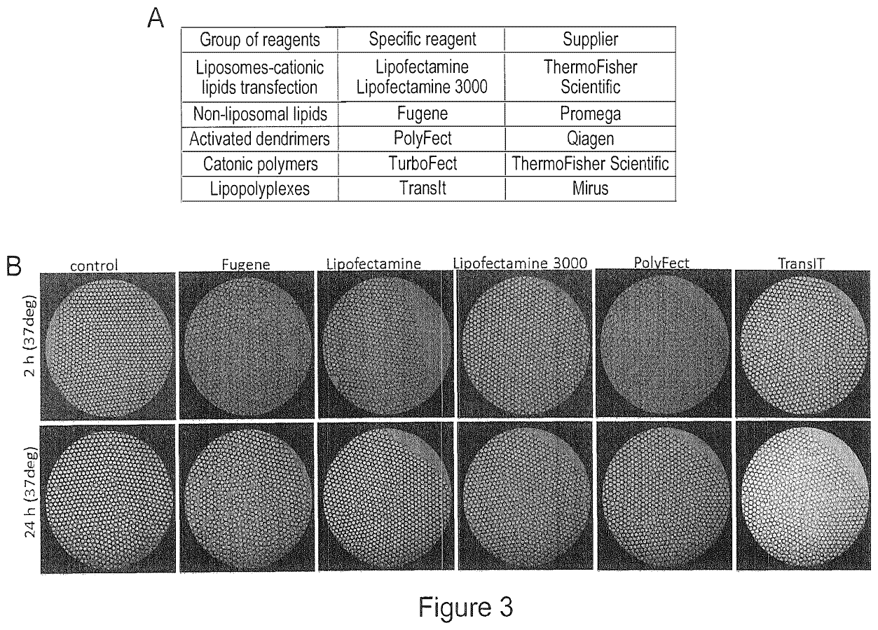

FIG. 3 shows that droplet generation and stability (24 h, 37 °C) is not affected by presence of different transfection reagents. FIG. 3a shows a list of transfection reagents, belonging to different subgroups. Picodroplets (300 pL) containing encapsulation media and different transfection reagents (1-5 L/sample) were tested immediately after generation (not shown) and following 24 h incubation at 37 °C (bottom images).

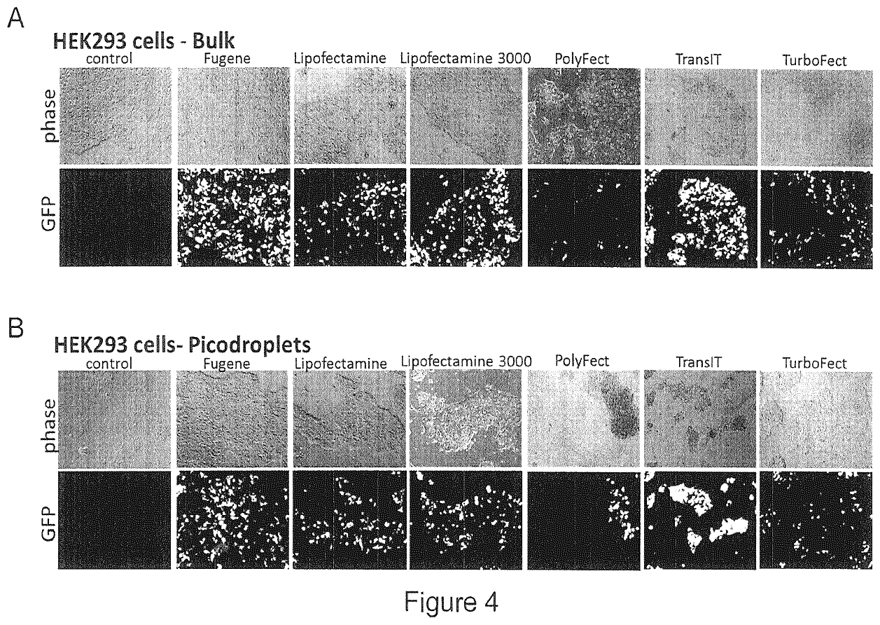

FIG. 4 shows that cells can be transfected in picodroplets using different transfection reagents. HEK293 cells were (1×10⁶ cell/ml) were mixed with 1 μg of GFP expressing plasmid, cells then were incubated in Eppendorf tubes (bulk; a) or in 300 pL picodroplets (picodroplets; b). Following 12 hours incubation, cells were seeded in 24-well plates and examined using fluorescence microscope 48 h post-transfection.

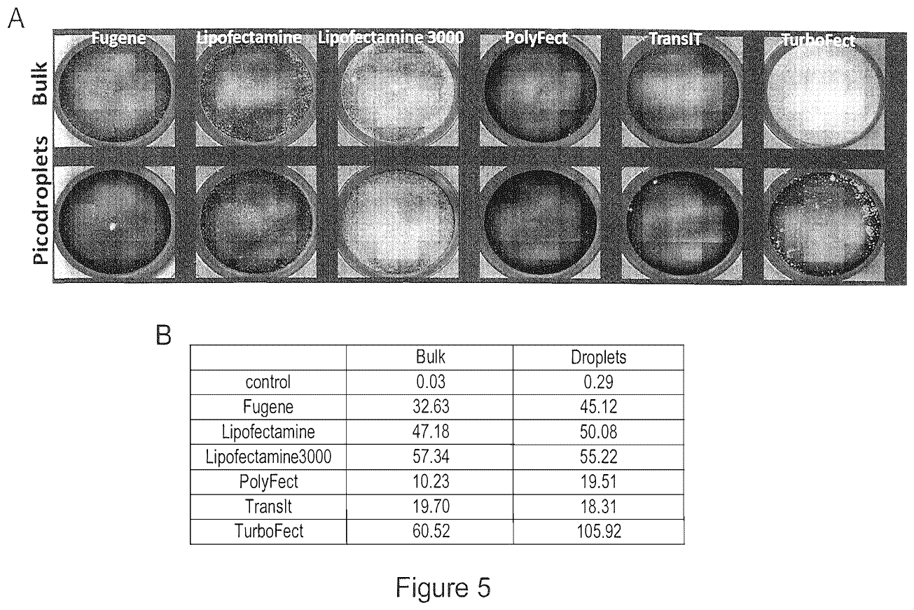

FIG. 5 shows the quantification of transfection efficiency of GFP expressing cells. HEK293 cells were (1×10⁶ cell/ml) were mixed with 1 μg of GFP expressing plasmid, cells then were incubated in Eppendorf tubes (bulk) or in 300 pL picodroplets (picodroplets). Following 12 hours incubation, cells were seeded in 24-well plates and examined using NyOne imaging 48 h post-transfection (a). Values measured by NyOne system (brightfield area and fluorescence are) were used to quantify percentage of transfected cells in each sample (b).

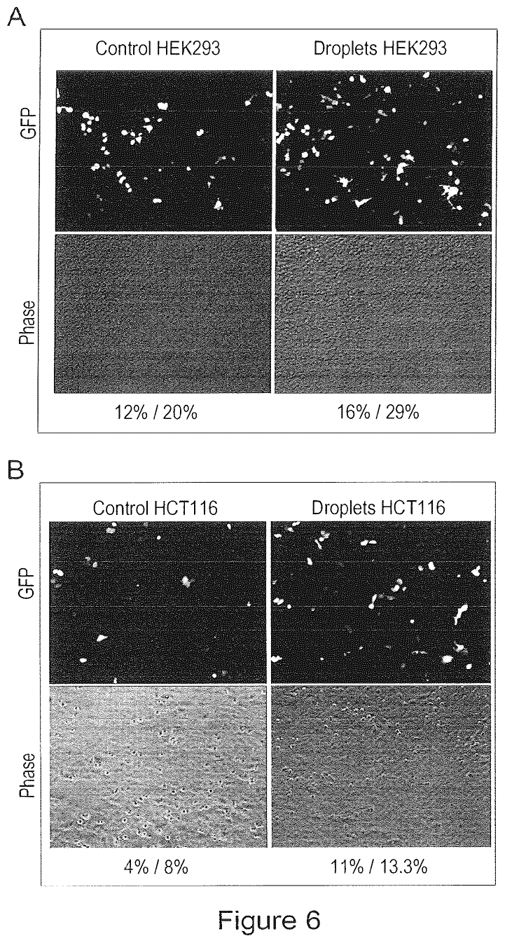

FIG. 6 shows a representative example of an experiment testing transfection efficiency of cells using a classical transfection method and our in-house developed method of transfection in picodroplets. HCT1116 cells or HEK293 cells (1.25×10⁵/well) were seeded into 24-well plate. Next day cells in 24-well plate (2.5×10⁵) were transfected with 0.25 μg DNA/Lipofectamine 3000 (ratio 1:2). For transfection in picodroplets, 2.5×10⁵ cells were encapsulated with encapsulation media and 0.25 μg/Lipofectamine 3000 (ratio 1:2). Following incubation of 2 hours, cells in 24-well plate were washed twice and media was replaced with fresh growth media. Cells in picodroplets were retained, pelleted using centrifugation, washed twice and re-seeded in fresh growth media. Transfection efficiency was imaged 24 hours post-transfection and then quantified 48 h post-transfection using NucleoCounter NC300 or NyOne.

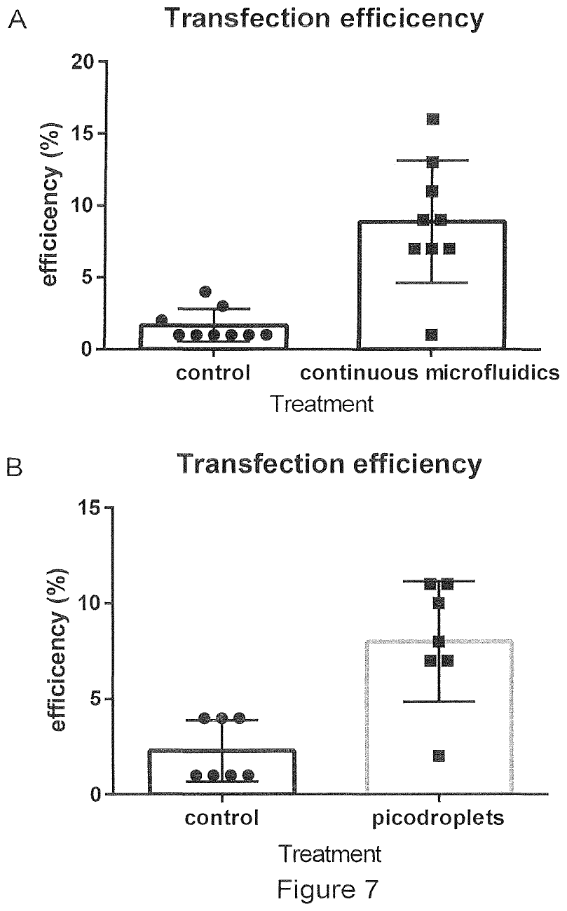

FIG. 7 shows a summary of experiments testing transfection efficiency using classical transfection method our in-house developed method of transfection in microfluidic system (picodroplets or continuous microfluidics). HCT1116 cells or HEK293 cells were transfected using classical transfection protocols (control) or using SF developed protocol for transfection in picodroplets/continuous microfluidic system. Transfection efficiency was quantified 48 h post-transfection using NucleoCounter NC300 or NyOne. Graphs represent mean−/+SD of 5 experiments done on continuous microfluidics (a, iBidi)) or 6 experiments performed in picodroplets (b).

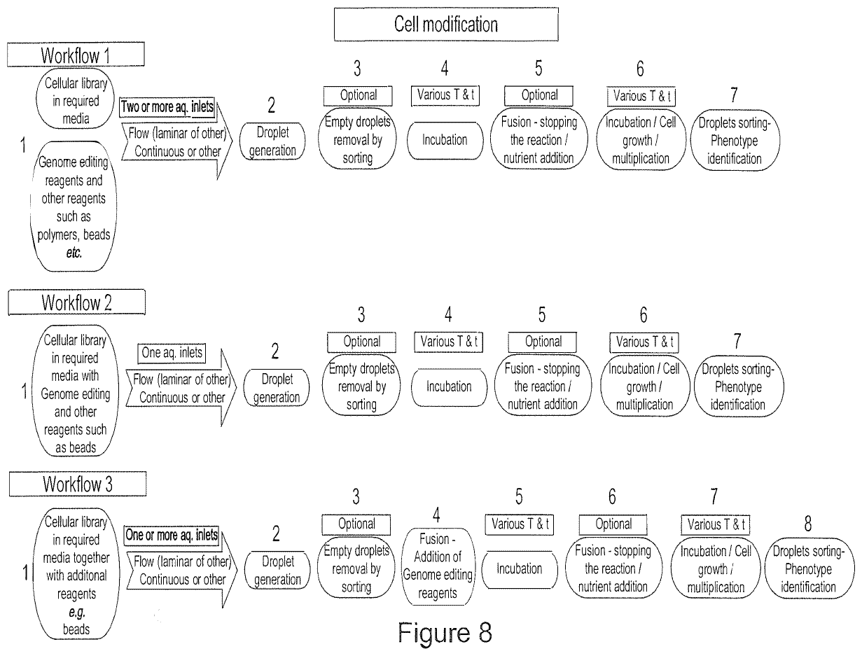

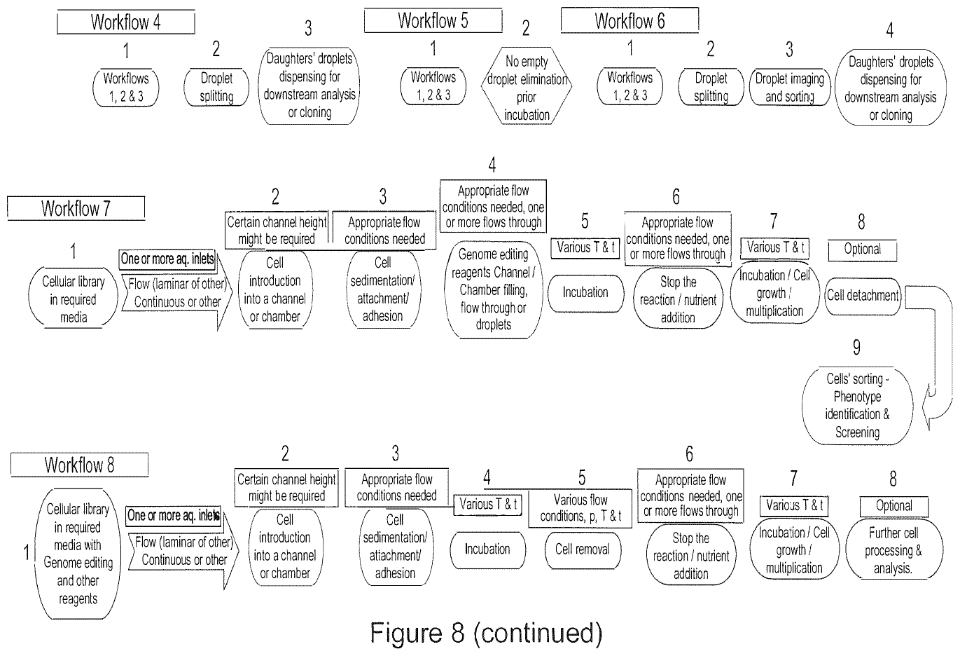

FIG. 8 shows a number of different workflows according to example implementations of the invention.

In workflow 1 the microfluidic system comprises at least two inlets, the first comprising a cellular library (preferably in suspension), optionally with a suitable growth media and a second inlet comprising biomolecules, such as genome editing reagents as well as other reagents, such as beads and polymers. In this example, the two inlets are fluidly connected to a first droplet generation device to generate at least one microfluidic droplet. In the next step, the droplets are sorted to remove droplets containing no cells before incubation (step four). Optionally in step 5, the droplets may be fused with at least one further droplet comprising reagents to stop a biological reaction and/or additional nutrients. Alternatively, the reagents and/or nutrients can be added or metered into a passing droplet. In step 6 the droplet is incubated, preferably for sufficient time for cell growth and division to occur, before finally, in step 7, the droplets are sorted according to their phenotype.

In workflow 2, a single inlet comprising the cellular library (preferably in suspension), optionally growth media, and biomolecules, such as genome editing reagents as well as optionally, other reagents, such as beads and polymers are fluidly connected to a first droplet generation device, Steps 3 to 7 are the same as workflow 1.

In workflow 3, a first single inlet comprising only the cellular library (preferably in suspension), optionally with growth media as well as optionally, other reagents, such as beads and polymers is fluidly connected to a first droplet generation device. The resulting droplet is analysed and sorted for the absence of cells. As in the preceding workflows, droplets lacking cells (or comprising multiple cells) may be passed to a waste outlet. In step 4, the first droplet is fused with at least a second droplet comprising biomolecules such as genome editing reagents. Steps 5 to 8 are the same as steps 4 to 7 of workflow 1.

Workflow 4 follows on from workflows 1, 2 or 3 and comprises a further step, step 2 (of workflow 4) of splitting the droplets following phenotype analysis into at least a first and second daughter droplet. In step 3 (of workflow 4) the daughter droplets are dispensed for further analysis or cloning.

Workflow 5 is based on either workflow 1, 2 or 3 but does not comprise the step of sorting, analysing and eliminating droplets that comprise no (or equally a multiple number of) cells.

Workflow 6 follows on from workflows 1, 2 or 3 and comprises the following further steps—step 2 (of workflow 6) of splitting the droplets following phenotype analysis into at least a first and second daughter droplet; step 3 (of workflow 6) of imaging and sorting the at least first and second daughter droplets before step 4 (of workflow 6), of dispending the daughter droplets for further analysis or cloning.

Workflow 7 is similar to workflow 3, but in step 1 the cellular library is an adherent cell culture. Accordingly, in workflow 7 at least one inlet comprising the cellular library (preferably an adherent cell culture), optionally with growth media is fluidly connected to a channel or chamber (step 2) where cell sedimentation, attachment or adhesion can occur (step 3). Steps 4 to 9 are the same as steps 4 to 8 of workflow 3. Workflow 7 may also comprise an additional step-step 8 of detaching the adhered cells before sorting and analysing the cell phenotype in step 9.

Workflow 8 is similar to workflow 1, but again the cellular library is adherent cell culture. Accordingly, in workflow 7 at least one inlet comprising the cellular library (preferably an adherent cell culture), optionally with growth media, and biomolecules, such as genome editing reagents as well as optionally, other reagents is fluidly connected to a channel or chamber (step 2) where cell sedimentation, attachment or adhesion can occur (step 3).

Steps 4 to 8 are the same as steps 4 to 7 of workflow 2. Workflow 8 may also comprise an additional step-step 5 of removing cells before further incubation and cell processing and analysis (step 8).

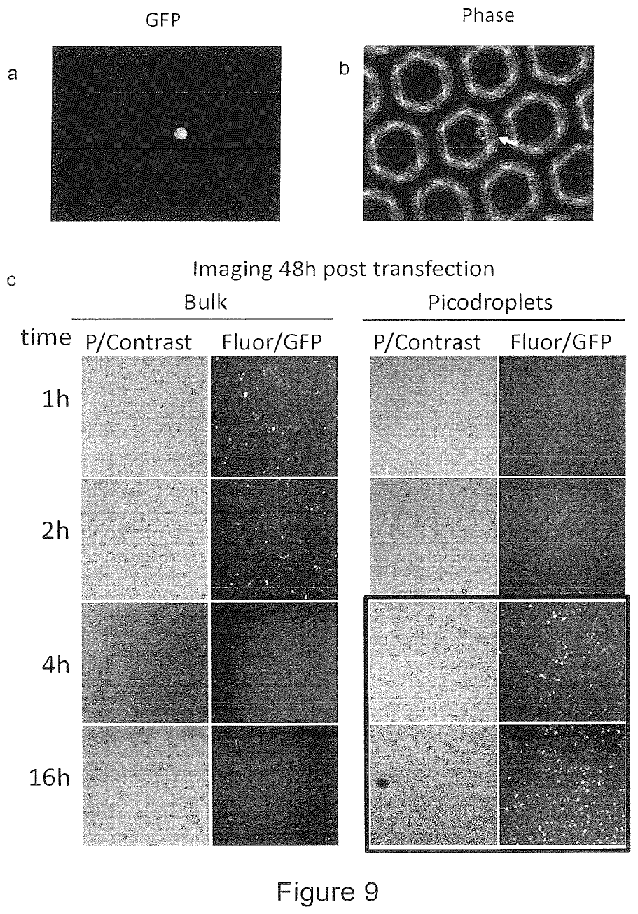

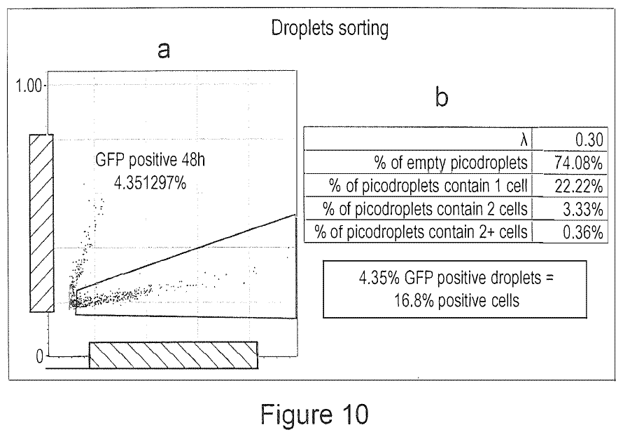

FIGS. 9 to 11 shows gene editing in droplets. HTC116 FIRE-line was used as a model cell-line and carried a with genome-inserted GFP sequence that was point mutated to stop protein expression. Following genome editing, the mutation is repaired resulting in the expression of a functional GFP protein.

### Experimental Procedure

HTC116 FIRE-line cells were encapsulated with genome editing reagents and transfection reagents (sgRNA 100 pM, ssODN 400 pM, DharmaFect 6 μl) in 400 pL picodroplets using Y-junction chip (no pre-mixing of cells and reagents prior to encapsulation into a single droplet). Cells were then incubated in picodroplets for 48 hours. Picodroplets were loaded into the system described herein (Cyto-Mine and analysed using standard Cyto-Mine™ procedure). Successful genome editing was indicated by the switch on GFP fluorescence. Picodroplets containing GFP expressing cells were sorted based on fluorescence signal and dispensed into 96-well plates. Cells outgrowth in 96-well plates was analysed 2 weeks post dispensing.

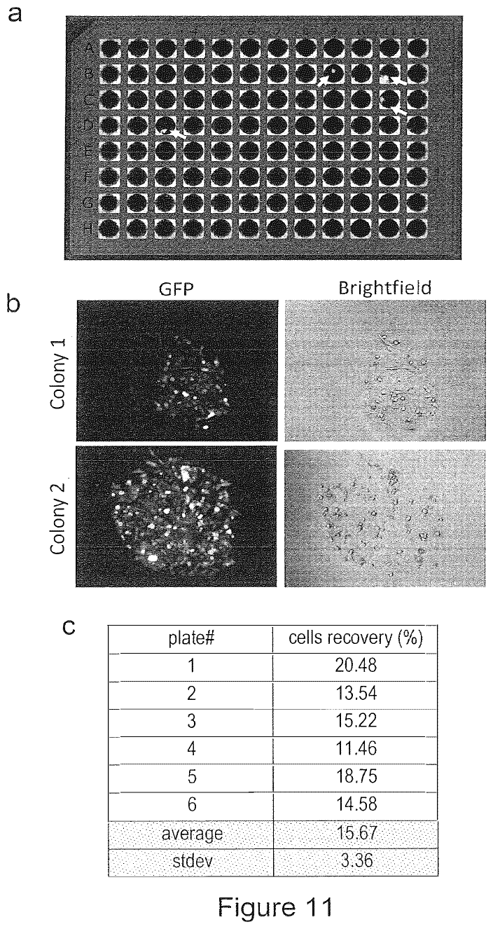

As shown in FIGS. 9 to 11, genome editing could be successfully performed in droplets, as shown by the expression of GFP. In particular, FIG. 9 shows that genome editing in droplets performs at least comparably to standard, bulk genome editing. Furthermore, as shown in FIG. 11, cells dispensed from the system of the invention grow and form colonies.

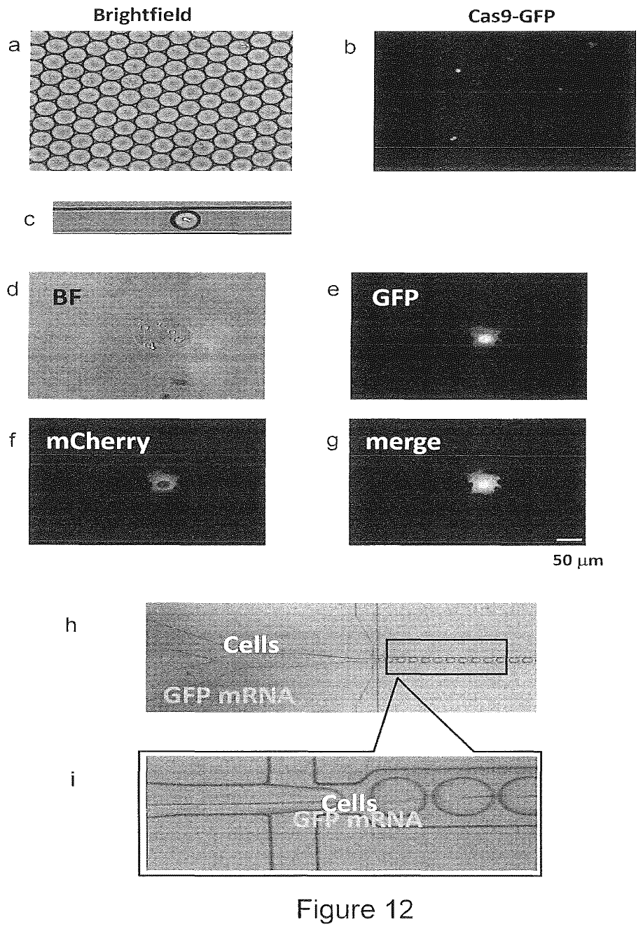

FIG. 12 shows the transfection of HCT116 cells with viral vectors expressing genome editing reagents, and in particular GFP tagged Cas9. 400 pL picodroplets containing HCT116 cells transfected with the viral vector were incubated for 18 hours, and then analysed for GFP expression. As shown in FIG. 12b a number of encapsulated cells were successfully transfected with Cas9-GFP.

### REFERENCES

1. Tomas Cermak, Erin L. Doyle, Michelle Christian, Li Wang, Yong Zhang, Clarice Schmidt, Joshua A. Baller, Nikunj V. Somia, Adam J. Bogdanove & Daniel F. Voytas. Efficient design and assembly of custom TALEN and other TAL-effector-based constructs for DNA targeting, Nucleic Acids Research 2011, 39(12) e82.
2. MV Wiles, W Qin, AW Cheng & H Wang. CRISPR-Cas9-mediated genome editing and guide RNA design. Mammalian Genome 2015, 26(9-10) p501-510.
3. Alexis C. Komor, Yongjoo B. Kim, Michael S. Packer, John A. Zuris & David R. Liu. Programmable editing of a target base in genomic DNA without double-stranded DNA cleavage, Nature 2016, 533(7603) p420-424.
4. Keiji Nishida, Takayuki Arazoe, Nozomu Yachie, Satomi Banno, Mayura Tabata, Masao Mochizuki, Aya Miyabe, Michihiro Araki, Kiyotaka Y. Hara, Zenpei Shimantani & Akihiko Kondo. Targeted nucleotide editing using hybrid prokaryotic and vertebrate adaptive immune systems. Science 2016, 353(6305) p1-14.
5. Martin P. Stewart, Armon Sharei, Xiaoyun Ding, Gaurav Sahay, Robert Langer & Klavs F. Jensen. In vitro and ex vivo strategies for intracellular delivery, Nature 2006, 538 p183-192.
6. Melinda G. Simon & Abraham P. Lee. Chapter 2 Microfluidic Droplet Manipulations and Their Applications. Microdroplet Technology: Principles and Emerging Applications in Biology and Chemistry 2012, p21-50, ISBN 978-1-4614-3264-7.

1.-71. (canceled)

72. A method of reacting a biomolecule with a single cell, the method comprising:
(i) providing a plurality of cells in a first fluid;
(ii) providing a plurality of biomolecules in a second fluid;
(iii) preparing microfluidic droplets from the first fluid and the second fluid, wherein the first fluid and second fluid are combined to form one fluid, and the microfluidic droplets are formed from the combined fluid; determining whether the microfluidic droplets contain no cells, one cell, or a plurality of cells, and sorting the droplets on the basis of the determination, wherein droplets comprising a single cell and at least one biomolecule are selected;
(iv) culturing the droplets selected in (iii) for sufficient time to perform a reaction between the single cell and the at least one biomolecule; and
(v) analyzing the droplets of (iv) for a predetermined property; wherein the method further comprises sorting the droplets dependent on the analysis.

73. The method of claim 72, wherein the method further comprises passing droplets with no or a plurality of cells to a waste outlet.

74. The method of claim 72, wherein the method further comprises splitting said droplet into at least a first and second daughter droplet.

75. The method of claim 72, wherein the droplets are cultured for at least 24 hours.
